(19) United States
(12) Patent Application Publication
(10) Pub. No.: US 2003/0133878 A1
(43) Pub. Date: Jul. 17, 2003

(54) Composition for the treatment of legionella pneumophila and a method for such treatment

(75) Inventor: Gilbert Theo Hinze, Randburg (ZA)

Correspondence Address: NATH & ASSOCIATES PLLC, 6th Floor, 1030 15th Street, NW, Washington, DC, 20005, US

(73) Assignee: RADICAL WATERS IP (PTY) LTD, Erasmuskloof (ZA)

(21) Appl. No.: 10/292,479

(22) Filed: Nov. 13, 2002

Related U.S. Application Data
(63) Continuation-in-part of application No. 09/529,723, filed on Jun. 26, 2000, now abandoned.
Application No. 09/529,723 is a 371 of international application No. PCT/US98/22371, filed on Oct. 23, 1998.

(30) Foreign Application Priority Data
Oct. 23, 1997 (ZA) 97/9485

(51) Int. Cl. A61L 9/04; A01N 39/00; A61K 33/40; A61K 33/14

(52) U.S. Cl. 424/45; 424/616; 424/662

(57) ABSTRACT

This invention relates to a composition and method for treating Legionella Pneumophila comprising an electro-chemically activated anion-contained aqueous solution.

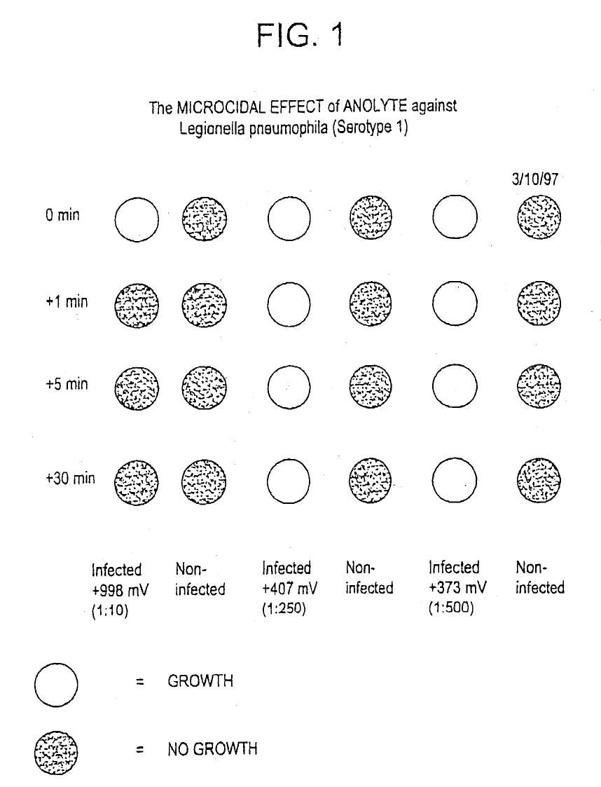

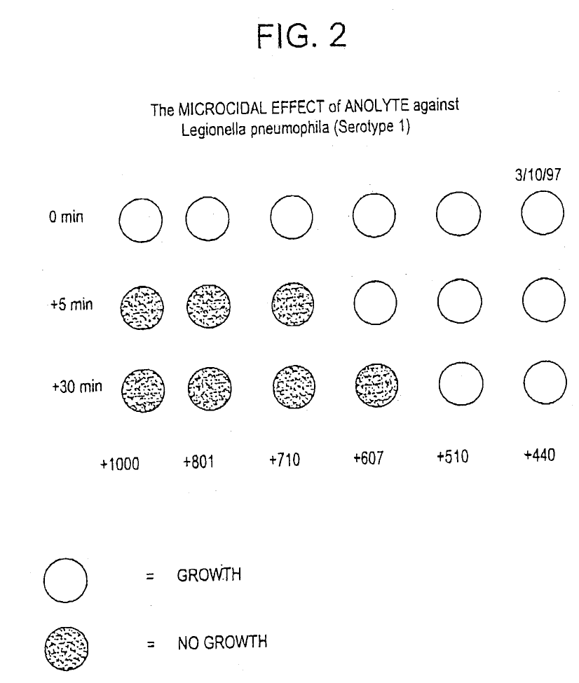

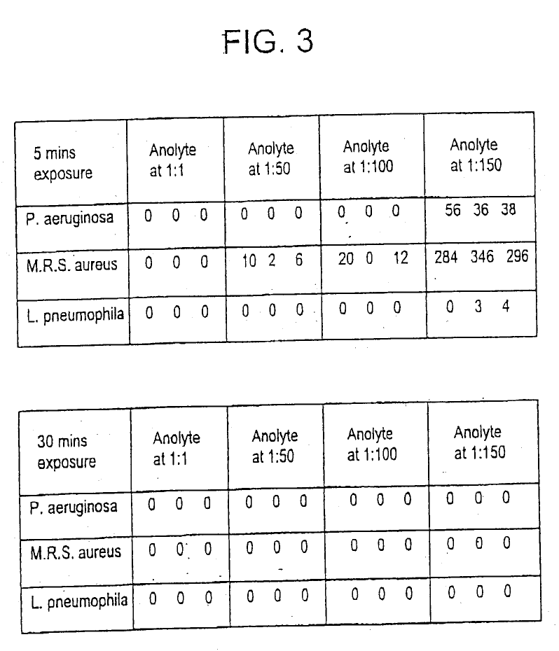

### FIELD OF THE INVENTION

This invention relates to a composition for the treatment of Legionella pneumophila , a method for treating Legionella pneumophila and the use of such composition in the preparation of a medicament for treating Legionella pneumophila.

### BACKGROUND TO THE INVENTION

So-called Legionnaire's disease became known in 1976 after an outbreak of a serious respiratory disease, diagnosed as having been caused by Legionella pneumophila. Current treatment in humans includes Erythromycin with the addition of Rifampicin in non-responding cases.

Legionella bacteria have a wide natural distribution in water and their growth is promoted by other micro-organisms, including Pseudomonas species, which provide nutrients and protect them from adverse conditions, including the effect of biocidal treatment of water.

The Legionella bacteria can infect humans by means of an aerosol, moving into the breathing zone of persons and deposition of the aerosol into the lungs. Other sources of infection include recreational waters, residential and industrial waters, air-conditioning systems, humidifiers, respiratory therapy apparatus, dental water supply lines and resuscitation systems.

Current control measures against infection include super heating, hyper-chlorination and chlorine gasification. However, no operating, maintenance, cleaning and decontamination procedures presently exist that are generally regarded as safe work practices.

### OBJECT OF THE INVENTION

It is accordingly the object of this invention to provide a composition for treating Legionella as well as an associated method for treating same.

### BRIEF SUMMARY OF THE INVENTION

According to a first aspect of the invention there is provided a composition for treating Legionella Pneumophila comprising an electro-chemically activated anion-containing aqueous solution.

The anion-containing solution, or so-called anolyte, may be obtained from the electrolysis of an aqueous solution of a salt. The salt may be sodium chloride. In particular it may be non-iodated sodium chloride or potassium chloride.

The anion-containing solution and the associated cation-containing solution may be produced by an electro-chemical reactor or so-called electrolysis device. The electro-chemical reactor may include a through flow, electro-chemical cell having two co-axial cylindrical electrodes with a co-axial diaphragm between them so as to separate an annular inter electrode space into a catalytic and an analytic chamber.

The anolyte may have a redox potential of above +600 mV and preferably about +750 mV and may have a pH of about 6.5-7.5. The anolyte may include any one of more or radical anion species from the group consisting of ClO; ClO⁻; HClO; OH⁻; HO₂⁻; H₂O₂; O₃; S₂O₈²⁻and Cl²O₆²⁻.

According to a second aspect of the invention there is provided a method for treating Legionella Pneumophila comprising the steps of atomising a suitable dosage of an electro-chemically activated, anion-containing aqueous solution; and dispensing the atomised dosage of aqueous anion-containing solution into an atmosphere to be treated, the aqueous solution being substantially as herein defined.

According to a third aspect of the invention there is provided the use of an electro-chemically activated anion-containing aqueous solution in the preparation of a medicament for use in the treatment of Legionella Pneumophila in humans.

### DETAILED DESCRIPTION OF PREFERRED EMBODIMENT

A preferred embodiment of the invention will now be described by means of three non-limiting examples only.

An electro-chemical reactor, may including a through flow, electrochemical cell having two co-axial cylindrical electrodes with a co-axial diaphragm between them so as to separate an annular inter-electrode space into a catalytic and an analytic chamber, was used to produce anolyte and catholyte solutions.

### EXAMPLE 1

Anolyte solution with varying characteristics was used as shown in the respective examples.

A series of trials have been conducted whereby various dilutions of aqueous anion-containing solutions have been seeded with Legionella pneumophila (Serotype 1) organisms and the microcidal effects of treatment with anolyte have been observed after incubation for a period of 4 days (96 hours) at a temperature of 37° C.

The efficacy of the treatment with anolyte at the various dilutions and times of exposure was established by the presence or absence of Legionella cultures on the infected BCYE culture medium.

Three replicates of each of the dilutions and of the control groups were seeded with a pure culture of Legionella Pneumophila (Serotype 1), resulting in counts of above 7 million parts per millilitre (TNTC).

Samples were collected at pre-determined time intervals and transferred onto the growth medium before being incubated for 4 days (96 hours) at ±37° C.

As can be seen from the Table for Example 1, anolyte was microcidal at levels between +998 mV and +407 mV (i.o.w. at a dilution rate of more than 1-10).

### EXAMPLE 2

Further tests were then conducted to narrow down the ranges of efficacy using a reducing-oxidation potential (ORP) as the monitoring (measuring) and on a similar basis as set out in Example 1.

As is illustrated in the Table for Example 2, it is deduced that:

1. A contact time of about 5 minutes at about 750 mV and a contact time of about 30 minutes at about +607 mV is completely microcidal against Legionella Pneumophila (Serotype 1); and

2. The microcidal effect of anolyte is directly proportional to the ORP of the dilution.

### EXAMPLE 3

P.aeruginosa and S.aureus (Methicillin resistant) strains were cultured overnight on blood agar plates. Both of these strains were obtained from clinical specimens obtained during routine laboratory investigations at the General Hospital in Johannesburg, South Africa. The L. pneumophila strain was cultured for 3 days on BCYE agar as it is a slow-growing organism. This isolate was obtained from the ATCC (American type culture collection) reference stock cultures, designated ATCC 33155.

These plate cultures were used for preparation of the liquid suspensions in Ringer's solution.

A suitable inoculum of each of the 3 test strains was removed from the agar plates with a nichrome loop and emulsified in 1/40 strength Ringer's buffer. These were then homogenised in a vortex mixer (the 1/40 Ringer's buffer is suitable for diluting the fastidious Legionella as well as the S.aureus and P.aeruginosa). Using a 0.5 McFarland's standard opacity tube, which is the equivalent to 150 million organisms/ml, the capacity of the three cultures in suspension was adjusted to an opacity to give a final count (i.e. after adding to the Ringer's solution or Ringer's-anolyte solution) of approximately 1 million colony forming units per ml (1×10⁶ cfus per ml—called the “high count challenge). A second set was prepared with a 1/10 dilution (1×10⁵ cfus—called the “low count challenge”).

| | |
|---|---|
| 1:1 | 1 part Ringer's 1/40 + 1 part anolyte (2.0 ml + 2.0 ml) |
| 1:50 | 49 parts Ringer's 1/40 + 1 part anolyte. (4.9 + 2.0) |
| 1:100 | 1 part 1:5 anolyte + 1 part Ringer's (2.0 ml + 2.0 ml) |
| 1:150 | 1 part anolyte + 2 parts Ringer's (1.0 ml + 2.0 ml). |

These dilutions were distributed in 100 μl quantities in 5 ml disposable plastic test tubes in triplicate for each set of organisms.

A thiosulphate neutraliser was made up by adding 2 crystals per 10 ml (which is also the amount used in the British Public Health Service Laboratories or PHLS including that of John Lee's Legionella Unit) and distributed in 10 μl quantities in plastic disposable test tubes.

All cultures were pre-tested microbiologically to determine whether any effect such as a decrease in the number of viable organisms recovered would occur, using any of the reagents such as thiosulphate neutraliser or a 30 minute exposure to Ringer's buffer.

This test was done in triplicate as follows:

| | Test tube dilutions: | | | | |
|---|---|---|---|---|---|
| | ∘ | ∘ | ∘ | ∘ | ∘ |
| | Ringer's | 1:1 | 1:5 | 1:100 | 1:150 |
| 100 μl | only | anolyte | anolyte | anolyte | anolyte |

To each set of test tubes containing either the anolyte dilutions or plain Ringer's (i.e. the control), 1 drop (10 μl) of culture was added. As the same conditions were being applied to both the test and the control samples, no special calculation was required for volume adjustment from 10 μl to 110 μl when the culture was added.

From the Ringer's only control tube, a further 1/100 dilution in Ringer's was made at the appropriate time interval (see below) to facilitate counting, should the original plate count be too high to observe individual cfus.

At the appropriate time in intervals, (5 mins and 30 mins post-exposure) 10 μl of organism in anolyte dilution/Ringer's only (control) was removed and mixed with the 10 μl of thiosulphate neutraliser. This “mix” was seeded onto a petri dish (blood agar for the S.aureus and P.aeruginosa and BCYE for the Legionella). The plates were spread over the entire surface with a sterile nichrome spreader.

The blood agar plates were incubated for 48 hours at 37° C. aerobically and the BCYE plates at the same temperature for 5 days aerobically in a sealed jar with a very moist atmosphere.

Colonies were counted using a colony counter with a magnifying lens and a grid.

No significant difference in the number of cfus/ml of the untreated (control) organisms were obtained after (a) being left in Ringer's solution for a 30 minute period and (b) treatment with sodium thiosulphate when compared with counts taken immediately after preparation of the suspensions. Thus any drop in cfus was purely due to the effect of the anolyte.

The “high count challenge” dose gave the following numbers of cfus/ml:

| | |
|---|---|
| S. aureus | 3.4 10⁶ cfus/ml |
| P. aeruginosa | 1.2 × 10⁸ cfus/ml |
| L. pneumophila | 2.7 × 10⁶ cfus/ml |

The “low count challenge” dose gave the following numbers of cfus/ml:

| | |
|---|---|
| S. aureus | 2.9 × 10⁵ cfus/ml |
| P. aeruginosa | 2.2 × 10⁵ cfu\ml |
| L. pneumophila | 4.9 × 10⁵ cfu/ml |

All cultures with a concentration of 10⁵ cfus/ml showed no growth (became non-viable) after being exposed to any of the dilutions of anolyte (1:1, 1:50, 1:100, 1:150) for both the 5 and 30 minute periods.

The results of these cultures containing 10⁶ cfus/ml treated in the same manner with anolyte dilutions were as set in the Table for Example 3.

It is envisaged that the following methods of treatment could be used:

1. By dosing anolyte onto elements such as a condenser used in air-conditioning systems;

2. By fogging anolyte into air-conditioning ducts or into the atmosphere e.g. in an operation theatre, etc.; and

3. By patients inhaling fogged anolyte, thereby exposing the Legionella organism to the anolyte in the alveoli of the lungs.

It will be appreciated that many variations in detail are possible without departing from the scope and/or spirit of the invention as claimed in the claims hereinafter.

1. A method for killing Legionella pneumophila and/or preventing Legionella pneumophila contamination in recreational waters, residential and industrial waters, air-conditioning systems, humidifiers, respiratory therapy apparatus, resuscitation systems, or the like environments and damp surfaces, comprising the steps of electrochemically activating an aqueous solution such that the solution includes separable and both of an aqueous, mixed oxidant, predominantly anion-containing solution and an aqueous, mixed reductant, predominantly cation-containing solution; separating the aqueous, mixed oxidant, predominantly anion-containing solution from the aqueous, mixed reductant, predominantly cation-containing solution; and either dosing the aqueous, mixed oxidant, predominantly anion-containing solution into environments or onto elements and apparatus to be treated, or fogging the aqueous, mixed oxidant, predominantly anion-containing solution onto surfaces or into an atmosphere or air-conditioning ducts.

2. The method as claimed in claim 1 wherein the electrochemically activated, aqueous predominantly anion-containing solution is prepared by means of electrolysis of an aqueous solution of a salt.

3. The method as claimed in claim 1 wherein the predominantly anion-containing solution and the predominantly cation-containing solution is produced by an electrolysis device, having a through-flow electrochemical cell with two co-axial cylindrical electrodes, with a co-axial diaphragm between the two electrodes so as to separate an annular inter-electrode space into a catholytic and an anolytic chamber; and wherein the anion-containing solution is separated from the cation-containing solution during production.

4. The method as claimed in claim 1 wherein the anion-containing solution is produced at a redox potential of above +600 mV and pH of between about 6.5 and 7.5.

5. The method as claimed in claim 1 wherein the electrochemically activated, aqueous solution is produced from an aqueous NaCl or KCl₂ solution, electrolysed to produce the mixed reductant and mixed oxidant species.

6. The method as claimed in claim 1 wherein the anion-containing solution includes mixed oxidant species selected from the group consisting of ClO; ClO⁻; HClO; OH⁻; HO₂⁻; H₂O₂; O₃; S₂O₈²⁻ and Cl₂O₆²⁻.

7. The method as claimed in claim 1 wherein the aqueous, mixed oxidant, predominantly anion-containing solution is fogged, nebulised and/or evaporated at a minimum rate of 250 ml/m³/day and under such conditions that 50% or more of droplets of the anion-containing solution have an average droplet size of less than 25 μm.

8. A method for treating Legionella pneumophila in a human or animal body, the method comprising the steps of electrochemically activating an aqueous solution such that the solution includes separable and both of an aqueous, mixed oxidant, predominantly anion-containing solution and an aqueous, mixed reductant, predominantly cation-containing solution; separating the aqueous, mixed oxidant, predominantly anion-containing solution from the aqueous, mixed reductant, predominantly cation-containing solution; and applying the aqueous, mixed oxidant, predominantly anion-containing solution to the human or animal body through atomisation by means of nebulisation, fogging and/or evaporation of the anion-containing solution and specifically introducing the atomised anion-containing solution into lung alveoli of the human or animal body, for example through normal breathing.

9. The method as claimed in claim 8 characterised therein that nebulisation, fogging and/or evaporation of the anion-containing solution is conducted at a minimum rate of 250 ml/m³/day and under such conditions that 50% or more of droplets of the anion-containing solution reaching the airways of the affected human or animal body are less than 5 μm in diameter.

10. A method for treating Legionella pneumophila in a human or animal body comprising administering an aqueous, mixed oxidant, predominantly anion-containing solution to a human or animal in need thereof, wherein the mixed oxidant, predominantly anion-containing solution includes mixed oxidant species selected from the group consisting of ClO; ClO⁻; HClO; OH⁻; HO₂⁻; H₂O₂; O₃; S₂O₈²⁻ and Cl₂O₆²⁻.

11. A composition adapted for treating Legionella pneumophila comprising an aqueous, mixed oxidant, predominantly anion-containing solution produced from an aqueous NaCl or KCl₂ solution, having a redox potential of above +600 mV and pH of between about 6.5 and 7.5, and including mixed oxidant species selected from the group consisting of ClO; ClO⁻; HClO; OH⁻; HO₂⁻; H₂O₂; O₃; S₂O₈²⁻ and Cl₂O₆²⁻.
